(19) United States
(12) Patent Application Publication
(10) Pub. No.: US 2004/0000482 A1
(43) Pub. Date: Jan. 1, 2004

(54) Viral removal

(75) Inventors: Kailing Wang, Carlingford (AU); Thomas Turton, Lane Cove (AU); Brendon O'Rourke, Pleasure Point (AU); Michael Evtushenko, Homebush (AU); Hari Nair, Baulkham Hills (AU)

Correspondence Address: BAKER & MCKENZIE, 805 THIRD AVENUE, NEW YORK, NY, 10022, US

(73) Assignee: Gradipore Limited, Frenchs Forest (AU)

(21) Appl. No.: 10/388,308

(22) Filed: Mar. 13, 2003

Related U.S. Application Data
(63) Continuation-in-part of application No. 10/006,241, filed on Dec. 7, 2001.
Application No. 10/006,241 is a continuation-in-part of application No. 09/931,342, filed on Aug. 16, 2001.
Application No. 09/931,342 is a continuation of application No. 09/470,823, filed on Dec. 23, 1999, now Pat. No. 6,464,851.

(30) Foreign Application Priority Data
Dec. 23, 1998 (AU) PP7906

(51) Int. Cl. G01N 27/26

(52) U.S. Cl. 204/518; 204/543

(57) ABSTRACT

Methods for removing a non-enveloped virus from a sample containing a target compound and non-enveloped virus contamination place the sample containing the target compound in a solvent chamber of an electrophoresis apparatus having a cathode and an anode, and a first solvent chamber being separated from a second solvent chamber by a separation barrier that has a defined pore size. The solvent chambers and the separation barrier are located between the cathode and anode.

A solvent is selected having a defined pH for the first solvent chamber, and a solvent is also added to the second solvent chamber. Applying an electric potential between the cathode and anode separates at least a portion of target compound on one side of the barrier and virus on the other side of the barrier. This applying step is maintained until a desired purity of the target compound is achieved.

### CROSS-REFERENCE TO RELATED APPLICATIONS

This application is a continuation-in-part of application Ser. No. 10/006,241 filed Dec. 7, 2001 which is a continuation-in-part of application Ser. No. 09/931,342 which is a continuation of application Ser. No. 09/470,823 which claims priority from Australian provisional application no. PP7906 filed Dec. 23, 1998.

### TECHNICAL FIELD

The present claims relate to methods for the removal of biological contaminants, particularly removal of non-enveloped viral contaminants from biological preparations.

### BACKGROUND

The modern biotechnology industry is faced with problems concerning the processing of complex biological solutions which typically include proteins, nucleic acid molecules and complex sugars that are contaminated with unwanted biological materials. Contaminants include microorganisms such as bacteria, viruses or other biomolecules derived from microorganisms or the processing procedure.

Viruses are some of the smallest non-cellular organisms known. These simple parasites are composed of nucleic acid and a protein coat. Viruses are typically very small and range in size from 1.5×10⁻⁸ m to 5.0×10⁻⁵ m. Viruses depend on the host cells that they infect to reproduce by inserting their genetic material into the host, often taking over the host's function. An infected cell frequently produces more viral protein and genetic material than its usual products. Some viruses may remain dormant inside host cells. However, when a dormant virus is stimulated, it can enter the lytic phase where new viruses are formed. Self-assembly occurs and viruses burst out of the host cell, resulting in killing the cell and releasing new viruses to infect other cells. Viruses cause a number of diseases in humans including smallpox, the common cold, chicken pox, influenza, shingles, herpes, polio, rabies, Ebola, hanta fever, and AIDS. Some types of cancer have also been linked to viruses.

Contamination with virus is a major concern when purifying plasma proteins, such as IgG and human serum albumin (HSA). A contaminant virus can potentially infect a patient receiving the contaminated plasma products. A virus that infects bacteria is known as a phage, and they are readily detected by examining culture plates for cleared zones in a coating or lawn of bacteria. In recent years, numerous infectious agents including several newly discovered hepatitis viruses and the agents of transmissible spongiform encephalopathies (TSE) have been identified as potential threats to the safety of blood and plasma. Examples of infectious agents are the immunodeficiency viruses (HIV-1 and HIV-2), the hepatitis viruses (HAV, HBV and HCV) and the Parvovirus (B-19).

The risks of contamination from enveloped viruses such as HIV, HBV, HCV have been greatly reduced over the last decade due to the introduction of sensitive virus screening methods and the inclusion of viral inactivation/removal steps during the manufacturing process.

Typically, large lipophilic inactivation compounds are used to inactivate a replicating virus by piercing the outer membrane and stopping the replicating process. For example, an enveloped virus contains a fatty, lipid membrane surrounding its protein coat, which may be penetrated by a liphophilic inactivation compound. Viral filtration methods are also effective against the larger enveloped viruses.

Risks, however, still exist with non-enveloped viruses such as HAV and human Parvovirus B19, which are more resistant and not inactivated by heat and detergent treatments (SD). A non-enveloped virus is not protected by a liphophilic membrane as in an enveloped virus, and leaves exposed a tough outer shell that is difficult for inactivation compounds large in size to penetrate. A common characteristic of non-enveloped viruses is their resistance to most physico-chemical treatments.

The purification of viruses from biological solutions is often a long and cumbersome process especially when purifying blood proteins. The process is made more complex by the additional step of ensuring the product is “bug” free.

The Gradiflow™ Technology

Gradiflow™ is a preparative electrophoresis technology for macromolecule separation which utilizes tangential flow across a polyacrylamide membrane when a charge is applied across the membrane, and is described in commonly assigned U.S. Pat. Nos. 5,650,055; 6,328,869; 6,402,913; and 6,413,402. The general design of the Gradiflow™ system facilitates the purification of proteins and other macromolecules under near native conditions.

Typically, Gradiflow™ technology is bundled into a cartridge comprising three membranes housed in a system of specially engineered grids and gaskets which allow separation of macromolecules by charge and/or molecular weight. The system can also concentrate and desalt/dialyse at the same time. The multimodal nature of the system allows this technology to be used in a number of other areas especially in the production of biological components for medical use.

Various techniques are known for purifying proteins, such as those described by commonly assigned U.S. Pat. Nos. 5,650,055; 6,328,869; 6,402,913; 6,413,402; and 6,464,851.

### SUMMARY

The present claims provide a method for removing a non-enveloped virus from a sample containing a target compound and non-enveloped virus contamination.

The sample containing the target compound is placed in a solvent chamber of an electrophoresis apparatus having a cathode and an anode, and a first solvent chamber being separated from a second solvent chamber by a separation barrier that has a defined pore size. The solvent chambers and the separation barrier are located between the cathode and anode.

A solvent is selected having a defined pH for the first solvent chamber, and a solvent is also added to the second solvent chamber. Applying an electric potential between the cathode and anode separates at least a portion of target compound on one side of the barrier and virus on the other side of the barrier. This applying step is maintained until a desired purity of the target compound is achieved.

In another aspect, applying an electric potential between the cathode and the anode moves at least a portion of the target compound through the barrier into the second solvent chamber while virus is substantially retained in the first solvent chamber or the barrier itself. Virus is substantially prevented from entering the second solvent chamber.

In another aspect, applying an electric potential between the cathode and anode moves virus through the barrier into the second solvent chamber while the target compound is substantially retained in the first solvent chamber.

In another aspect, target compound that enters the membrane moves back into the first solvent chamber upon periodically stopping and reversing the electric potential, thus preventing virus from re-entering the first solvent chamber.

These and other features of the claims will be appreciated from review of the following detailed description of the application along with the accompanying figures.

### BRIEF DESCRIPTION OF THE DRAWINGS

FIG. 1 is a block diagram of a method for removing non-enveloped virus from a sample containing a target compound;

FIG. 2 is a PAGE analysis of albumin purification from human plasma during simultaneous HAV removal using Tris Borate buffer pH 9.0 under forward polarity;

FIG. 3 shows PAGE analysis of IgG purification from human plasma during simultaneous HAV removal using Mes/Bis Tris buffer pH 5.4 under reverse polarity.

FIG. 4 shows a 4 to 20% native electrophoresis gel of samples and a Western blot of samples;

FIG. 5 shows a 4 to 20% native electrophoresis gel of samples and a Western blot of samples;

FIG. 6 shows samples from up and downstream were taken at time intervals (x-axis) during the isolation of albumin from plasma;

FIG. 7 shows samples from the second phase of an IgG separation were taken from both up- and downstreams (U/S and D/S respectively) at 30 minute intervals. The samples were assayed for endotoxin using a LAL Chromogenic assay (Cape Cod Assoc.)

FIG. 8 shows samples taken from up- and downstream at 30 minute intervals during a 90 minute purification (x-axis); and

FIG. 9 shows four to 25% native gel electrophoresis of samples from an HSA purification from endotoxin spiked plasma.

### DETAILED DESCRIPTION

Embodiments for separating viral contaminants from biological samples according to the present claims are described in non-limiting detail below.

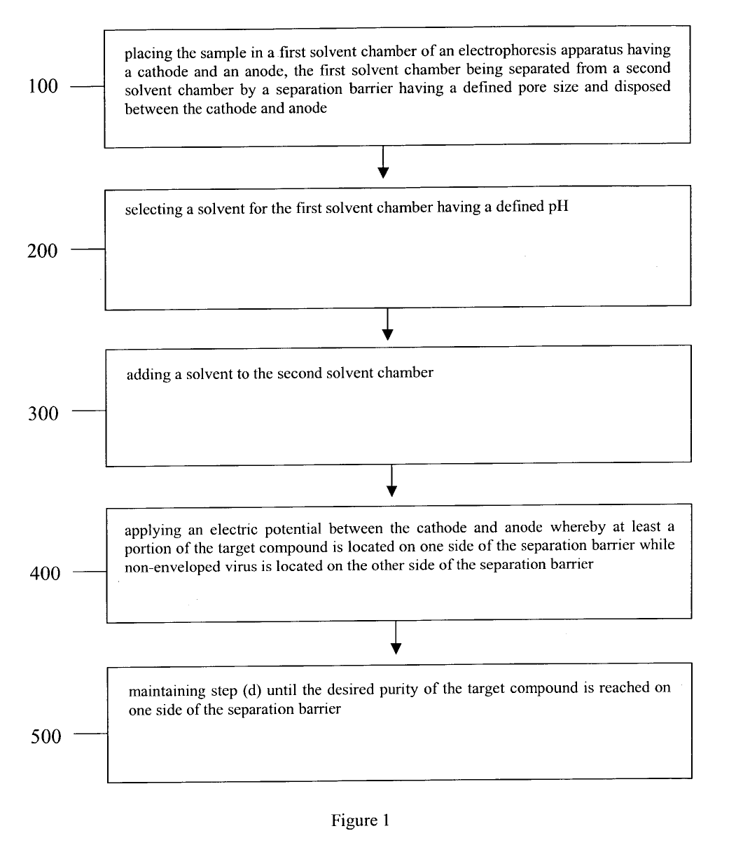

FIG. 1 refers to a block diagram of a method for removing a non-enveloped virus from a sample containing a target compound in accordance with one aspect of the present claims. Typically, the non-enveloped virus is one or more of immunodeficiency virus, hepatitis virus, adenovirus, reovirus, rhinovirus, papillomaviruns, foot and mouth disease virus, or parvovirus. Non-limiting examples of immunodeficiency virus are human immunodeficiency virus 1 or human immunodeficiency virus 2. Non-limiting examples of hepatitis virus are hepatitis A virus, hepatitis B virus or hepatitis C virus. A non-limiting example of parvovirus is Parvovirus B-19. However, other non-enveloped viruses characterized by the absence of a membrane surrounding its protein coat, may also be separated from biological samples in accordance with the present claims.

The methods described herein are suitable for removing non-enveloped virus from samples containing a variety of biological compounds such as blood proteins, immunoglobulins, peptides, and recombinant proteins, among other target compounds. For example, proteins, peptides, antibodies, growth factor, immunoglobulin, immunoglobulin G, lactalbumin, lactoglobulin, transgenically expressed recombinant proteins or peptides, recombinant forms thereof, and mixtures thereof are representative and non-limiting examples of target compounds that may be purified of viral components. Non-limiting examples of recombinant proteins include fibrinogen, albumin, antibodies, or insulin. During removal of non-enveloped virus, other biological contaminants may be removed from the sample using the present claims as well. For example, the present claims are also suitable for removing other biological contaminants such as lipopolysaccharides, pathogens, toxins, infectious agents, and endotoxins, prions, bacteria, fungi, yeasts, or protozoa in addition to removing virus.

Block 100 depicts placing a sample in a first solvent chamber of an electrophoresis apparatus. A suitable electrophoresis apparatus contains a cathode and an anode, with at least a first and second solvent chamber. The first solvent chamber is separated from the second solvent chamber by a separation barrier having a defined pore size. The selection of a defined pore size is based on the type of target compound and type of contaminant to be separated and is readily ascertainable by the skilled practitioner. The solvent chambers are disposed between the cathode and the anode. Examples of suitable electrophoresis apparatus are found in U.S. Pat. Nos. 6,413,402, 6,328,869, and 5,650,055, and are incorporated by reference herein. Other electrophoresis apparatus having an anode, cathode, solvent chambers, and an separation barrier are known and are also suitable to practice the present claims.

In one embodiment, the separation barrier is a membrane. Typically, the membrane has a molecular mass cut-off close to the apparent molecular mass of the target compound. In one embodiment, the membrane has a molecular mass cut-off of between about 3 and 1000 kDa. In another embodiment, the membrane has a molecular mass cut-off of at least about 3 kDa. One of ordinary skill in the art will appreciate that other membrane chemistries or constituents may be used. For example, the separation barrier may be a gel.

Block 200 selects a solvent for the first solvent chamber. In one embodiment, the solvent has pH lower than the isoelectric point of the target compound. In another embodiment, the solvent has a pH at the isoelectric point of the target compound, and in another embodiment the selected solvent has a pH above the isoelectric point of the target compound. The selection of an appropriate solvent and solvent pH for the first solvent chamber is based on the type of target compound and type of contaminant to be separated, and whether the target compound is isolated in the first or second solvent chamber. The selection of an appropriate solvent is readily ascertainable by one of ordinary skill in the art.

Block 300 adds a solvent to the second solvent chamber. In one embodiment, the solvent has pH lower than the isoelectric point of the target compound. In another embodiment, the solvent has a pH at the isoelectric point of the target compound, and in another embodiment the selected solvent has a pH above the isoelectric point of the target compound. Similar to block 200, the selection of an appropriate solvent and solvent pH for the second solvent chamber is based on the type of target compound and type of contaminant to be separated, and whether the target compound is isolated in the first or second solvent chamber. The selection of an appropriate solvent is readily ascertainable by one of ordinary skill in the art.

Block 400 applies an electric potential between the cathode and anode, separating at least a portion of target compound on one side of the barrier and virus on the other side of the barrier. More than one target compound may be isolated within a given separation. For example, viral contaminant may be removed from a mixture of two or more proteins or other biological compounds. Similarly, the target compound may be separated from two or more non-enveloped viruses. Another non-limiting example would be the removal of non-enveloped virus and endotoxins.

In one embodiment, block 400 applies an electric potential between the cathode and anode whereby at least a portion of the target compound moves through the barrier into the second solvent chamber while virus is substantially retained in the first solvent chamber or the barrier. In another embodiment, block 400 applies an electric potential between the cathode and anode whereby the virus moves through the barrier into the second solvent chamber while the target compound is substantially retained in the first solvent chamber or the barrier.

Selection or application of the voltage and/or current applied varies depending on the separation. In one embodiment, the typical electric potential may reach as high as approximately 300 volts. However, in other embodiments, depending on transfer, efficiency, scale-up and particular method, near 0 V to 5000 V are used. Higher voltages may be used, depending on the apparatus and sample to be treated. Selection of a suitable voltage is readily ascertainable by practitioners in the art.

Optionally, the electric potential may be periodically stopped and reversed to move virus or other contaminant having entered the barrier to move back into the volume or stream from which it came, while substantially preventing the target compound that has passed completely through the barrier to pass back through the barrier.

In one embodiment, the target compound is collected or removed from the second solvent chamber. In another embodiment, the target compound is collected or removed from the first solvent chamber. In one embodiment, solution in at least one of the solvent chambers or streams containing separated target compound is collected and replaced with suitable solvent to ensure that electrophoresis continues efficiently. In one embodiment, solvent and/or sample are passed through the solvent chambers to form streams, thus enabling the processing of large volumes of sample in an efficient and rapid manner. Suitable apparatus may also be adapted to accommodate large volume through-put as well as different separation configurations. Of course, those skilled in the art will appreciate that these collection methods and arrangements of the electrophoresis apparatus are known and readily ascertainable by those skilled in the art.

Block 500 maintains the potential applied in block 400 until the desired purity of the target compound is reached in one of the solvent chambers. A desired amount of target compound may be isolated and extracted before complete separation of any given sample is effected.

To assist in understanding the present claims, the following examples are included and describe the results of a series of experiments. The following examples relating to this application should not be construed to specifically limit the application or such variations of the application, now known or later developed, which fall within the scope of the application as described and claimed herein.

In the following examples, the term “stream 1(S1)” refers to the first solvent chamber volume. The term “stream 2(S2)” refers to the second solvent chamber. The term “forward polarity” is used when the first electrode is the cathode and the second electrode is the anode in the electrophoresis apparatus and current is applied accordingly. The term “reverse polarity” is used when polarity of the electrodes is reversed such that the first electrode becomes the anode and the second electrode becomes the cathode. The term “buffer” is intended to include solutions of electrolytes. The buffer is a solution that conducts electricity. The buffer maintains to some extent a pH of its environment.

### Analytical Methods

A range of model viruses were selected, based on the characteristics of potential viral contaminants of the blood product being tested (e.g. small size/large size, DNA/RNA, envelope/non-envelope; resistance to physical and chemical agents). Two different types of non-enveloped model viruses (Porcine Parvovirus (PPV) and Canine Parvovirus (CPV) for small DNA non-enveloped viruses; Hepatitis A virus (HAV) for small RNA non-enveloped viruses) were selected for viral clearance studies because they have a high resistance to inactivation processes and cannot be removed effectively by currently existing nanofiltration technique. Porcine Parvovirus (PPV) and Canine Parvovirus (CPV) were used as model viruses for pathogenic non-enveloped Parvovirus B19. Viral removal studies on the purification of albumin, IgG, fibrinogen and α1-protease inhibitor were performed by independently spiking human plasma with model viruses. The following experiments established methods for removing a non-enveloped virus from a sample containing a target compound.

Membrane-Based Electrophoresis

A number of membrane-based electrophoresis apparatus developed by Gradipore Limited, Australia were used in the following experiments. Description of membrane-based electrophoresis can be found in commonly assigned U.S. Pat. Nos. 5,039,386 and 5,650,055, and are incorporated herein by reference.

In summary, the apparatus typically included a cartridge which housed a number of membranes forming two chambers, cathode and anode connected to a suitable power supply, reservoirs for samples, buffers and electrolytes, pumps for passing samples, buffers and electrolytes, and cooling means to maintain samples, buffers and electrolytes at a required temperature during electrophoresis.

The cartridge contained three substantially planar membranes positioned and spaced relative to each other to form two chambers through which sample or solvent can be passed. A separation membrane was positioned between two outer membranes (termed restriction membranes as their molecular mass cut-offs are usually smaller than the cut off of the separation membrane). When the cartridge was installed in the apparatus, the restriction membranes were located adjacent to an electrode. The cartridge is described in U.S. Pat. No. 6,328,869, and is incorporated by reference herein. ps Polyacrylamide Gel Electrophoresis (PAGE)

Standard PAGE methods were employed as set out below.

Reagents: 10×SDS Glycine running buffer (Gradipore Limited, Australia), dilute using Milli-Q water to 1× for use; 1×SDS Glycine running buffer (29 g Trizma base, 144 g Glycine, 10 g SDS, make up in RO water to 1.0 L); 10×TBE II running buffer (Gradipore), dilute using Milli-Q water to 1× for use; 1×TBE II running buffer (10.8 g Trizma base, 5.5 g Boric acid, 0.75 g EDTA, make up in RO water to 1.0 L); 2×SDS sample buffer (4.0 ml, 10% (w/v) SDS electrophoresis grade, 2.0 ml Glycerol, 1.0 ml 0.1% (w/v) Bromophenol blue, 2.5 ml 0.5M Tris-HCl, pH 6.8, make up in RO water up to 10 ml); 2×Native sample buffer (10% (v/v) 10 ×TBE II, 20% (v/v)PEG 200, 0.1 g/L Xylene cyanole, 0.1 g/L Bromophenol blue, make up in RO water to 100%); Coomassie blue stain (Gradipure™, Gradipore Limited). Note: contains methanol 6% Acetic Acid solution for de-stain.

Molecular weight markers (Recommended to store at −20° C.): SDS PAGE (e.g. Sigma wide range); Western Blotting (e.g. color/ rainbow markers).

SDS PAGE with Non-reduced Samples

To prepare the samples for running, 2×SDS sample buffer was added to sample at a 1:1 ratio (usually 50 μL/50 μL) in the microtiter plate wells or 1.5 ml tubes. The samples were incubated for 5 minutes at approximately 100° C. Gel cassettes were clipped onto the gel support with wells facing in, and placed in the tank. If only running one gel on a support, a blank cassette or plastic plate was clipped onto the other side of the support.

Sufficient 1×SDS glycine running buffer was poured into the inner tank of the gel support to cover the sample wells. The outer tank was filled to a level approximately midway up the gel cassette. Using a transfer pipette, the sample wells were rinsed with the running buffer to remove air bubbles and to displace any storage buffer and residual polyacrylamide.

Wells were loaded with a minimum of 5 ml of marker and the prepared samples (maximum of 40 μl). After placing the lid on the tank and connecting leads to the power supply the gel was run at 150V for 90 minutes. The gels were removed from the tank as soon as possible after the completion of running, before staining or using for another procedure (e.g. Western blot).

Staining and De-staining of Gels

The gel cassette was opened to remove the gel which was placed into a container or sealable plastic bag. The gel was thoroughly rinsed with tap water, and drained from the container. Coomassie blue stain (approximately 100 ml Gradipure™, Gradipore Limited, Australia)) was added and the container or bag sealed. Major bands were visible in 10 minutes but for maximum intensity, stained overnight. To de-stain the gel, the stain was drained off from the container.

The container and gel were rinsed with tap water to remove residual stain. 6% acetic acid (approximately 100 ml) was poured into the container and sealed. The de-stain was left for as long as it takes to achieve the desired level of de-staining (usually 12 hours). Once at the desired level, the acetic acid was drained and the gel rinsed with tap water.

PPV Removal During Plasma Protein Purifications

Cell Lines

The minipig kidney cell line (MPK, ECACC 87032604, Life Technologies) was maintained in Dulbecco's modified Eagle medium (DMEM) supplemented with 20% fetal bovine serum (FBS) and 4 mM L-glutamine. Monolayer cultures were grown in a 37° C. humidified incubator with 5% (v/v) CO₂ and were subcultured by dissociating the monolayer with 0.25% trypsin/0.02% EDTA (JRH Biosciences). All tissue culture reagents were purchased from Thermo Trace, Australia, except where indicated.

Propagation ofPPV

PPV strain NADL-2 (ATCC VR 742) was propagated in cultures of MPK cells. Monolayers of MPK cells (approximately 70% confluent) were inoculated with PPV in DMEM supplemented with 5% FBS and after absorption for 2 hours, media was added to a final volume of 40 ml per 182 cm² flask. The cultures were incubated until cytopathic effects (CPE) were greater than 90%. PPV-infected MPK cell cultures were freeze/thawed three times and centrifuged at 3,000 g for 15 min at 4° C. The supernatant was aliquoted into cryovials and frozen at −80° C. Titers of up to 10⁷ were obtained in the stock preparation which was used for spiking diluted plasma at a ratio of 1:10.

PPV Infectivity Assay

PPV infectivity was assessed by a TCID₅₀ assay in MPK cells. Flat-bottom 96-well plates, seeded with MPK cells, were inoculated 1-2 days later with ten-fold dilutions of PPV virus stock or PPV-spiked samples (filtered through a 0.2 μm filter) and incubated at 37° C. in 5% CO₂ for 10-14 days when the wells were examined for CPE. Six replicates were included for each dilution. Virus titers were calculated as TCID₅₀ using the method of Reed and Muench. These assays were performed in 12-well plates with a 2 ml inoculum.

PPV PCR Assay

DNaseI Treatment and DNA Extraction

Two units of DNaseI (Promega) was added to 180 μl of each sample and incubated at 37° C. for 1 hr in buffer containing 40 mM Tris-HCI (pH 8.0), 10 mM MgSO₄ and 1 mM CaCl₂ (Promega). The reaction was stopped with 20 mM EGTA (pH 8.0). The DNA from DnaseI-treated samples was extracted using phenol-chloroform and DNA was ethanol precipitated according to Sambrook et al. (1989). Extracted DNA was serially diluted 1/10 in H2O and four replicates of each dilution were subjected to the nested PCR.

Nested PCR

Detection of PPV was performed using a nested polymerase chain reaction (PCR) assay adapted from the protocol of Soares et al. (1999). Two outer primers

P1 5′-ATACAATTCTATTTCATGGGCCAGC-3′ and

P6 5′-TATGTTCTGGTCTTTCCTCGCATC-3′ were used initially to amplify a 330 bp sequence. Primers designed internal to this fragment

P2 5′-TTGGTAATGTTGGTTGCTACAATGC-3′ and

P5 5′-ACCTGAACATATGGCTTTGAATTGG-3′ were used in the second reaction to yield a 127 bp fragment. Amplifications were done in a DNA thermal cycler (icycler, BioRad). The first reaction was subjected to 95° C. for 5 min prior to 30 cycles at 95° C./15 s, 55° C./15 s and 72° C./10 s. In the second PCR reaction, initial denaturation was 95° C. for 5 min followed by 30 cycles at 95° C./15 s, 55° C./15 s and 72° C./3 s. PCR reactions included the final concentrations of 500 nM of each primer, 200 μM of each dNTP, 1.5 mM MgCl₂, MBI fermentas reaction buffer (10 mM Tris HCl pH 8.8, 50 mM KCl, 0.08% Nonidet P40) and 2.5 U of Taq (MBI fermentas). In the first reaction 5 μl of extracted DNA was used as template and the second reaction contained 5 μl of amplicon from the initial reaction.

Ten μl of product from the second PCR reaction was subjected to electrophoresis on a 10% polyacrylamide gel (BioRad). The gel was stained with 0.5 μg/ml ethidium bromide before visualising on a UV transilluminator. PCR titles were expressed as genomic equivalents.

CPV Removal During Plasma Protein Purifications

Propagation of CPV

CPV was propagated in culture of CRFK epithelial cell line and harvested using a standard freeze-thaw technique as described above in propagation of PPV.

CPV PCR Assays

The detection of CPV were perforned by PCR using the following primers:

| | |
|---|---|
| CPVVP2230 | 5′ GCAGTTAACGGAAACATGGC 3′ |
| CPVVP21230 | 5′ TCTCCTTCTGGATATCTTCC 3′ |

Amplifications were done in a DNA thermal cycler (icycler, BioRad). Initial denaturation was 95° C. for 5 min followed by 35 cycles at 95° C./5 m, 52° C./1 min and 72° C./1.5 min. PCR reactions included the final concentrations of 500 nM of each primer, 200 μM of each dNTP, 1.5 mM MgCl₂, MBI fermentas reaction buffer (10 mM Tris HCl pH 8.8, 50 mM KCl, 0.08% Nonidet P40) and 2.5 U of Taq (MBI fermentas). The amplification of CPV DNA produces a 1 kb fragment from a single reaction. PCR titres were expressed as genomic equivalents.

CPV Infectivity Assay

TCID₅₀ assays were stained and infectivity confirmed calorimetrically in 96 well plates with the use of CPV antibodies and HRP substrates (TropBio indirect immunoperoxidase kit for staining CPV infected cultures). Cytotoxicity and interference assays were required to determine sensitivity of assays. Staining was performed according to manufacturers instructions. Cell culture and TCID₅₀ assays were performed according to the standard procedures described above in PPV infectivity assay.

Hepatitis A Virus (HAV) Removal During Electrophoresis

Cell Lines

FRHK-4 cells (ATCC CRL-1688; passage 30) were maintained in Dulbecco's modified Eagle medium (DMEM) supplemented with 10% FBS and 4 mM L-glutamine. Monolayer cultures were grown in a 37° C. humidified incubator with 5% (v/v) CO₂ and were subcultured by dissociating the monolayer with 0.25% trypsin/0.02% EDTA (JRH Biosciences). All tissue culture reagents were purchased from Thermo Trace, Australia, except where indicated.

Propagation of HAV

Hepatitis A (product ATCC VR-1402, strain HM175) was propagated in cultures of FRHK-4 cells. Monolayers of FRHK-4 (approximately 70% confluent) were inoculated with HAV in DMEM supplemented with 5% FBS and after absorption for 2 hours, media was added to a final volume of 40 ml per 182 cm² flask. The cultures were incubated until cytopathic effects (CPE) were greater than 90%. HAV-infected FRHK 4 cell cultures were freeze/thawed three times and centrifuged at 3,000 g for 15 min at 4° C. The supernatant was aliquoted into cryovials and frozen at −80° C. Titres of HAV was determined by plaque assay as pfu/ml. In partitioning studies, viral loads were calculated as pfu/ml in the starting material and final pool products according to Barardi et al. (Barardi et al. 1999).

pfu/ml=average number of plaques per one dilution×dilution factor×inoculum (1 ml/inoculum in ml)

HAV Virus Analysis

RNA Extraction

Protocol followed for HAV RNA extraction supplied with the QIAmp®) Viral RNA Mini kit (QIAGEN). Serial dilutions performed with DEPC treated water.

RT-PCR

The method described by Monceyron, C. and Grinde, B. (1994) was adapted for HAV detection by PCR. Two outer primers HAVOS—5′ ttggttggatgaaaatggtt 3′ and HAVOA—5′ taagattcggagatgttggtc 3′ were used initially to amplify a 344 bp sequence. Primers designed internal to this fragment HAVIS—5′ caacctgtccaaaagatgaat 3′ and HAVIA—5′ aatccaggtttccatacaggt 3′ were used in the ‘nested’ reaction to yield a 158 bp fragment.

RT-PCR was performed using SUPERSCRIPT™ one step RT-PCR kit with Platinum® Taq (Invitrogen). PCR reaction mix was created using the supplied reaction mix at a concentration of 1× (containing a final concentration of 200 μM of each dNTP, 1.2 mM MgSO₄); an additional 1.5 mM MgSO₄ was added in addition to 200 μM of each dNTP, 200 nM of each required primer and 1 μl of RT/Platinum® Taq (Invitrogen) in a final volume of 50 μl. In the rt reaction, 2 μl of extracted DNA was used as template. DEPC treated Milli Q H₂O was used for a negative control.

Reverse transcription was performed in a thermal cycler (icycler, BioRad) at 50° C. for 20 min. A touchdown PCR protocol was then used for amplification of HAV cDNA as follows; initial denaturation at 95° C. for 4 min prior to 30 cycles at 95° C./15 s, 65-51° C./15 s (0.5° C. decrements) and 72° C./30 s followed by a further 15 cycles at 95° C./15 s, 51° C./15 s and 72° C./30 s.

The ‘nested’ reaction contained a final concentration of 1×reaction buffer (MBI fermentas, 10 mM Tris HCl (pH 8.8 at 25° C.), 50 mM KCl, 0.08% Nonidet P40), 200 μM of each dNTP, 1.5 mM MgCl₂, 200 nM of each required primer and 2.5 U of Taq (MBI fermentas) in a final volume of 50 μl adjusted with Milli Q H₂O. Two μl of amplicon from the initial reaction was used as template. The ‘nested’ reaction was performed in a thermal cycler (icycler BioRad) using the following protocol: initial denaturation at 95° C. for 5 min followed by 30 cycles at 95° C./15 s, 60° C./15 s and 72° C./15 s.

Ten μl of product from the second PCR reaction was subjected to electrophoresis on a 10% poly acrylamide gel (BioRad). The gel was stained with 0.5 μg/ml ethidium bromide before visualising on a UV transilluminator. PCR titres were expressed as log₁₀ genomic equivalents.

Infectivity Assay for HAV

HAV infectivity was assessed by a plaque assay in FRHK-4 cells. Twelve well plates, seeded with FRHK-4 cells, were inoculated with serial dilutions of HAV virus stock or HAV-spiked samples before and after electrophoresis. Inoculum was adsorbed for 90 minutes at 37° C. After incubation samples were removed from each well and replaced with 2 ml plaque assay media (2×MEM, 2%(v/v) FBS, 2 mM L-glutamine, 100 μM non-essential amino acids, 0.75% (v/v) CMC without phenol red). Plates were incubated at 37° C., 5% CO₂ until CPE was observed. Plaque assay media was removed and cells washed 3 times with PBS (w/o calcium or magnesium). One ml of Naphthalene Black solution (0.1% (w/v) Naphthalene Black prepared in 5% (v/v) acetic acid) was then added to each well and incubated for 20 min at room temperature with rocking. Stain was then removed and plates allowed to dry overnight prior to fixing. One ml of cold methanol (−20° C.) was added to each well and incubated for 5 min at RT. Methanol was removed and fixing step repeated before allowing plates to dry overnight. Plaques were counted and virus titre was calculated as pfu/ml described above (Barardi et al. 1999).

Electrophoresis Protein Separation Runs Spiked With Model Non-Enveloped Viruses

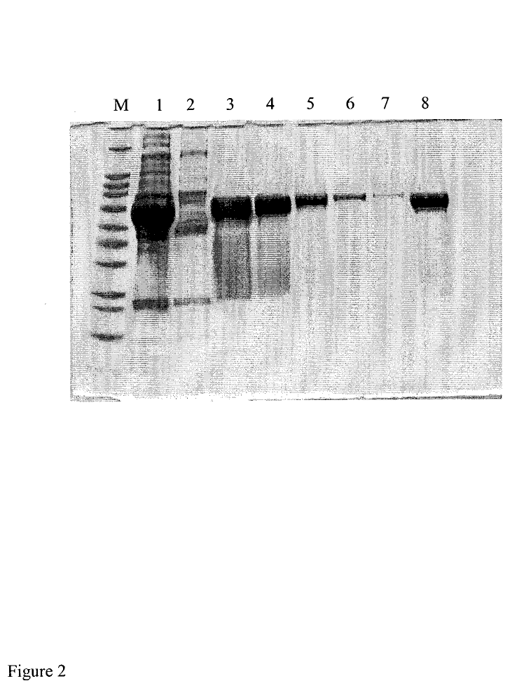

For albumin purification from plasma, a membrane cartridge, comprising a 150 kDa pore size separation membrane sandwiched between two 5 kDa pore size restriction membranes was used in the separation unit. Separations were performed using Tris-Borate buffer pH 9.0 ( 20 mM Boric Acid, 45 mM Tris) and an electric potential of 250 volts, with the positive electrode configured at stream 1 (S1), was applied across the membrane separation unit. The separation was analyzed by PAGE analysis and illustrated in FIG. 2. Lane M: MW Marker, Lane 1: 1 in 3 diluted human plasma spiked with 10%( v/v) HAV HM175, Lane 2: Albumin depleted plasma, Lanes 3-7: Purified albumin product from hourly harvests, Lane 8: Pooled Albumin product (1 ml of each harvest combined).

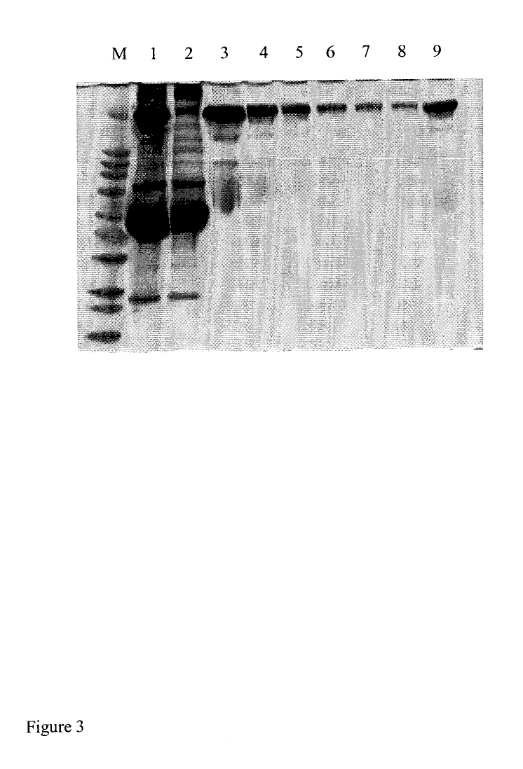

For IgG purification from plasma, a membrane cartridge, comprising a 1000 kDa pore size separation membrane sandwiched between a 5 kDa and a 80 kDa pore size restriction membranes was used in the separation unit of an electrophoresis apparatus produced by Gradipore Limited under the Gradiflow™ name. Separations were performed using Mes-Bis Tris pH 5.4 (41 mM MES, 6.3 mM Bis Tris, 0.1% (v/v) Tween 20) buffer and an electric potential of 250 volts, with the positive electrode configured at S1, was applied across the membrane sandwich to perform the electrophoresis. The separation was analyzed by PAGE analysis and illustrated in FIG. 3. Lane M: MW Marker, Lane 1: 1 in 3 diluted human plasma spiked with 10%( v/v) HAV HM175, Lane 2: IgG depleted plasma, Lanes 3-8: Purified IgG product from hourly harvests, Lane 9: Pooled IgG product (1 ml of each harvest combined).

For α1-proteinase inhibitor purification from plasma, a membrane cartridge, comprising a 80 kDa pore size separation membrane sandwiched between two 5 kDa pore size restriction membranes was used in the separation unit. Separations were performed using Tris-Borate buffer pH 9.0 ( 20 mM Boric Acid, 45 mM Tris) and an electric potential of 250 volts, with the positive electrode configured at S1, was applied across the membrane separation unit.

For fibrinogen purification from plasma, a membrane cartridge, comprising a 1000 kDa pore size separation membrane sandwiched between two 5 kDa pore size restriction membranes was used in the separation unit. Separations were performed using Tris-Borate buffer pH 9.0 ( 20 mM Boric Acid, 45 mM Tris) and an electric potential of 250 volts, with the positive electrode configured at S1, was applied across the membrane separation unit.

Frozen pooled plasma (CliniSys Associates, USA) was thawed at 37° C. An aliquot (1.5 ml) of PPV virus supernatant was added to 5 ml of plasma and the volume made up to 15 ml with buffer. PPV-spiked plasma was placed in S1 and the product in S2 was harvested at 60, 120, 240 and 360 min. After each harvest, 10 ml of buffer was used to replenish S2.

Calculation of Virus Reduction

The virus reduction factor for an individual purification is defined as the log₁₀ of the ratio of the virus load in the pre-purification material (spiked starting material) divided by the virus present in the post purification material (Output material). This enabled a reduction factor to be calculated.

$${10}^{R} = \frac{{Volume}_{1} \times Titre per unit input volume}{{Volume}_{2} \times Titre per unit output volume}$$

Where R=reduction factor.

The formula takes into account both the titres and volumes of the materials before and after the purification step.

### Results

Membranes of different pore sizes were configured to either restrict or facilitate movement of viruses. Samples before and after each process were quantified for the level of virus by nested PCR/RT-PCR and TCID₅₀ infectivity assays. Complete viral removal was achieved by restricting the pore size of separation membrane and/or inducing an appropriate charge on the virus. Table 1, 2 and 3 show the summary of viral clearance results. These results have demonstrated that the separation barrier-based electrophoresis apparatus used has the capability of remove small non-enveloped viruses effectively.

TABLE 1
PPV clearance during blood product separation by electrophoresis

| | Nested PCR | TCID₅₀ Infectivity |
|---|---|---|
| Blood Product | (Viral Log reduction) | (Viral Log reduction) |
| Albumin | >6 | >6 |
| α1-PI | >4 | >4 |
| IgG | >6 | >6 |
| Hemoglobin | >7 | >5.6 |

TABLE 2
CPV clearance during plasma product separation by electrophoresis

| | PCR | TCID₅₀ Infectivity |
|---|---|---|
| Plasma Product | (Viral Log reduction) | (Viral Log reduction) |
| Albumin | 4.8 | 4.3 |
| α1-PI | 4.8 | 4.3 |
| IgG | 4.3-4.4 | 4.1 |
| Fibrinogen | 3.7-4.0 | n/a |

TABLE 3
HAV clearance during plasma product separation by electrophoresis
(Virus titres were determined as Genome equivalents/ml by PCR or
pfu Units/ml by infectivity assay)

| | RT PCR | Infectivity |
|---|---|---|
| Plasma Product | (Viral log reduction) | (Viral log reduction) |
| Albumin | >5 | >6 |
| IgG | >6 | >6 |

The following experiment establish that other biological contaminants may also be removed according to the present claims.

Enveloped-Virus Removal During Plasma Protein Purification

IgG Purification Procedure

Platelet free plasma was diluted one part in three with Tris-borate, pH 9.0 running buffer and placed in stream 1 and spiked with either Lambda or T7 phage to a concentration of approximately 10⁸ pfu/mL (plaque forming units/mL). A potential of 250V was placed across a separating membrane with a molecular weight cut off of 200 kDa and with 3 kDa restriction membranes. A membrane of this size restricts IgG migration while allowing smaller molecular weight contaminants to pass through the membrane, leaving IgG and other large molecular weight compounds in the stream 1. A second purification phase was carried out using a GABA/Acetic acid buffer, pH 4.6 with a 500 kDa cut off separating membrane and with 3 kDa restriction membranes. A potential of 250V reversed polarity was placed across the system resulting in IgG migration through the separation membrane leaving other high molecular weight contaminants stream.

Examination of samples taken at 30 minutes intervals was made on reduced SDS-PAGE 4-20% gels. One hundred and fifty microliter samples were taken at each time point sample and mixed with 100 μL of appropriate Escherichia coli culture (Strain HB101 was used for T7 and strain JM11 for Lambda). The mixtures were incubated for 15 minutes at 37° C. and then added to 2.5 mL of freshly prepared molten soft agar, and vortexed. The mixtures were poured over culture plates of Luria Agar, and incubated at 37° C. overnight. The plates were inspected for the presence of virus colonies (plaques) in the lawn of E. coli and the number of plaques was recorded. If the virus had infected the entire E. coli population, the result was recorded as confluent lysis.

HSA Purification Procedure

Pooled normal plasma was diluted one in three with Tris-Borate (TB) 10 running buffer, pH 9.0 and spiked with approximately 10⁸ pfu/mL of Lambda or T7 phage. The mixture was placed in stream 1 of the electrophoresis apparatus. Albumin was isolated from platelet free plasma in a one-phase process using the charge of albumin at a pH above its isoelectric point (pI) and its molecular weight. Thus a cartridge having a 75 kDa cutoff separation membrane and two 50 kDa restriction membranes was used. The albumin was removed from high molecular weight contaminants by its migration through the separation membrane while small molecular weight contaminants dissipated through the 50 kDa restriction membrane. Samples were taken at regular intervals throughout a 90 minute run.

The presence of the purified HSA in the stream 2 was demonstrated by examination by SDS-PAGE. Virus was detected as previously described in the IgG purification procedure.

Fibrinogen Purification Procedure

Cryo-precipitate 1, produced by thawing frozen plasma at 4° C. overnight was removed from plasma by centrifugation at 10000 g at 4° C. for 5 minutes. The precipitate was re-dissolved in Tris-Borate buffer (pH 9.0) and placed in stream 1 of the electrophoresis apparatus. Stream 1 was spiked with either Lambda or T7 phage to a concentration of approximately 10⁸ pfu/mL. A potential of 250V was applied across a cartridge having a 300 kDa separation membrane for a period of 2 hours. Stream 2 was replaced with fresh buffer at 30 minute intervals. A second cartridge was then inserted having a 500 kDa cutoff separation membrane. A second phase was used to concentrate the fibrinogen through the second cartridge 100 at pH 9.0. Stream 2 was harvested at 60 minutes. The product was dialyzed against PBS pH 7.2 and analyzed for clotting activity by the addition of calcium and thrombin (final concentrations 10 mM and 10 NIG unit/mL respectively).

The presence of purified fibrinogen was confirmed by examination on reduced SDS PAGE 4-20% gels. The presence of either T7 or Lambda in the time point samples was tested using the previously described method.

Prion Removal

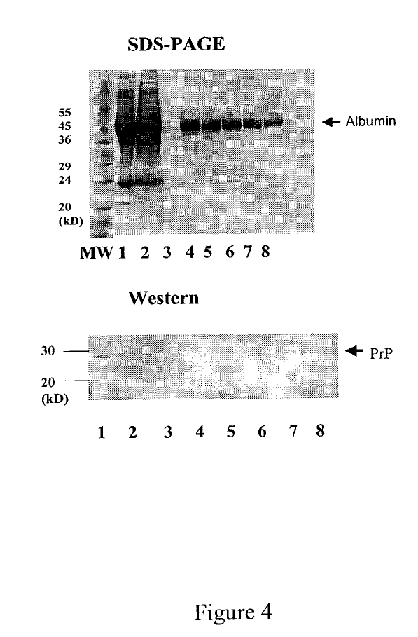

Human plasma (⅓ ratio), were mixed with bovine brain homogenate, containing PrP^{C} and placed in stream 1 of an electrophoresis apparatus. Purification of albumin was performed at 250 V using a cartridge with a separation membrane of 150 kDa and two restriction membranes of 5 kDa and 20 mM Tris-Borate (TB) running buffer, pH 9.0. Stream 2 fractions were collected every 60 minutes over a 5-hour run. The running conditions were selected such that the running buffer pH was higher than albumin pI, but lower than pI of PrP^{C} and the separation of albumin and PrP^{C} was achieved based on their charge differences. The presence of purified albumin in stream 2 was examined by SDS-PAGE and the yield was measured using a Bromocresol Green Assay (Trace Scientific). Anti-PrP Western blot, used to detect PrP^{C}, showed that PrP^{C} remained in stream 1 and stream 2 albumin fractions were completely free of PrP^{C} as shown in FIG. 4. Lanes 1 and 2 of both the electrophoresis gel and the Western blot show stream 1 at 0 minutes (human plasma with bovine brain homogenate) and at 300 minutes (albumin depleted human plasma) respectively. Lanes 3 through 8 show stream 2 and 0 minutes, 60 minutes (albumin), 120 minutes, 180 minutes, 240 minutes, and 300 minutes, respectively.

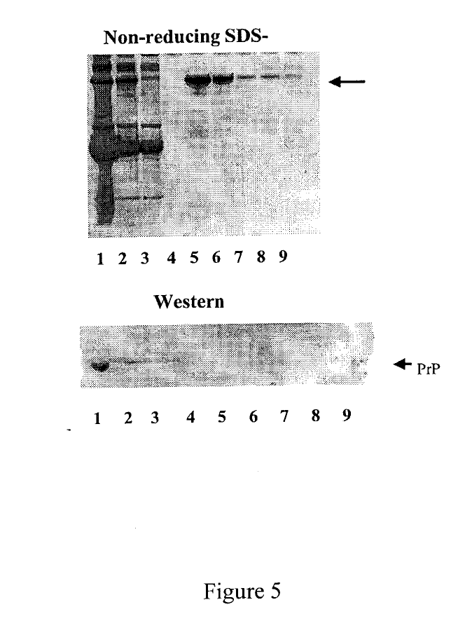

Similar partitioning experiments were carried out in the purification of Immunoglobulin from human plasma. Human plasma (⅓ ratio) were mixed with bovine brain homogenate, containing PrP^{C} and placed in stream 1 of a separation apparatus. By using an 800 kDa separation membrane, 5 kDa and 80 kDa-restriction membranes cartridge and 30 mM GABA/Acetic Acid (pH 4.6), the spiked bovine PrP^{C} was completely removed from stream 2 fractions which contained the purified human Immunoglobulin as shown in FIG. 5. Lanes 1, 2, and 3 of both the electrophoresis gel and Western blot show stream 1 and 0 minutes (human plasma with bovine brain homogenate), at 240 minutes (albumin depleted human plasma), and at 300 minutes (IgG depleted human plasma) respectively. Lanes 4 through 9 show stream 2 at 0 minutes, 60 minutes (IgG), 120 minutes, 180 minutes, 240 minutes, and 300 minutes, respectively. The separation of IgG and PrP^{C} was achieved based on their size differences.

Albumin Quantitation

Fifty microliters of sample from each time point were diluted with 50 μL of PBS buffer. A 20 μL aliquot of each diluted sample was placed in a microplate well. A standard curve with a maximum concentration of 40 mg/mL albumin was prepared using PBS as the diluent. The standard curve dilutions were also placed in the microplate (2T1 plasma/well). The bromocresol green reagent was added to all the wells (200 uL/well) and the absorbance at 630 nm was read using a Versamax microplate reader. A standard curve was drawn on a linear scale and the concentration of albumin in stream 1 and stream 2 samples were read from the curve. The volume in the appropriate stream 1 or stream 2 at the time of sampling was multiplied by the concentration of each sample, thus providing a value for the total HSA present in each stream.

Albumin was transferred to the stream 2 and was detected in the BCG assay (FIG. 1), and visualized on a 4-20% SDS polyacrylamide electrophoresis gel. By Western blot analysis PrP^{C} was detected in stream 1 and no prion was detected in the stream 2 samples.

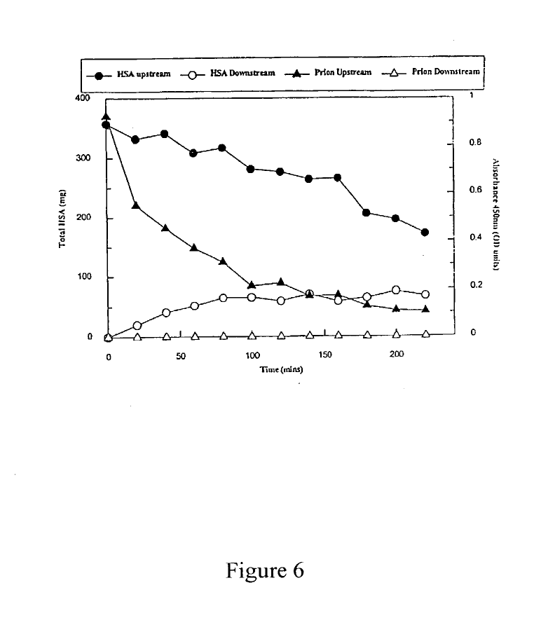

Referring to FIG. 6, samples from stream 1 and stream 2 were taken at time intervals (x-axis) during the isolation of albumin from plasma. Albumin was measured in the samples by mixing with BCG reagent and reading the absorbance of 630 nm. The concentration of albumin in each sample was calculated from the standard curve, and multiplied by the volume of stream 1 or stream 2 to obtain the total HSA in stream 1 or stream 2 (y-axis). All samples were assayed for prion using a sandwich ELISA, and recording the absorbance values at 450nm (second y-axis).

Prion Detection by Anti-PrP Western Blot

After subjecting the samples to SDS-PAGE analysis, a semi-dry transfer of proteins onto the nitrocellulose (NC) was performed for 1 hour at 15V. The NC was then blocked at 37° C. for 30 minutes before being incubated with anti-PrP antibody, R029, (Prionics, Switzerland) and subsequently incubated with HRP-conjugated secondary antibody. Western blot was developed by Enhanced Chemiluminescence ECL™ (Amersham Pharmacia Biotech).

Endotoxin Removal During Plasma Protein Purification

IgG Purification Procedure

Platelet free plasma was diluted one part in three with Tris-borate, pH 9.0 running buffer and placed in stream 1 of an electrophoresis apparatus and spiked with purified E. coli endotoxin to a concentration of 600 EU/mL (endotoxin units/mL). A potential of 250V was placed across a cartridge 100 having a separating membrane with a molecular weight cut off of 75 kDa restriction membranes with a molecular weight cut off of 50 kDa. A separation membrane of this size restricts IgG migration whilst allowing smaller molecular weight contaminants to pass through the membrane, leaving IgG and other large molecular weight compounds in the stream 1. A second purification phase was carried out using a MES/bis-tris buffer, pH 5.4 with a cartridge having a separating membrane with a molecular weight cut off of 500 kDa restriction membranes with a molecular weight cut off of 80 kDa. A potential of 250V reversed polarity was placed across the system resulting in IgG migration through the separation membrane leaving other high molecular weight contaminants in stream 1.

Examination of samples taken at 30 minutes intervals was made on reduced SDS-PAGE 4-20% gels. Endotoxin was tested for by using a LAL Pyrochrome Chromogenic assay purchased from Cape Cod Associates. All samples were appropriately diluted and the endotoxin assay was performed according to the manufacturer instructions. Stream 1 and stream 2 samples taken at 30 minute intervals during the second phase of an IgG purification from endotoxin spiked plasma were tested for endotoxin using a LAL Chromogenic assay. The results shown in FIG. 5 demonstrate that the endotoxin was almost entirely found in the stream 1 at all time points. As noted above, Lanes 1, 2, and 3 of both the electrophoresis gel and Western blot show stream 1 and 0 minutes (human plasma with bovine brain homogenate), at 240 minutes (albumin depleted human plasma), and at 300 minutes (IgG depleted human plasma) respectively. Lanes 4 through 9 show stream 2 at 0 minutes, 60 minutes (IgG), 120 minutes, 180 minutes, 240 minutes, and 300 minutes, respectively. Stream 2 contained only 0.7% of the initial endotoxin. Reduced SDS-PAGE examination showed that IgG had been successfully isolated in the stream 2.

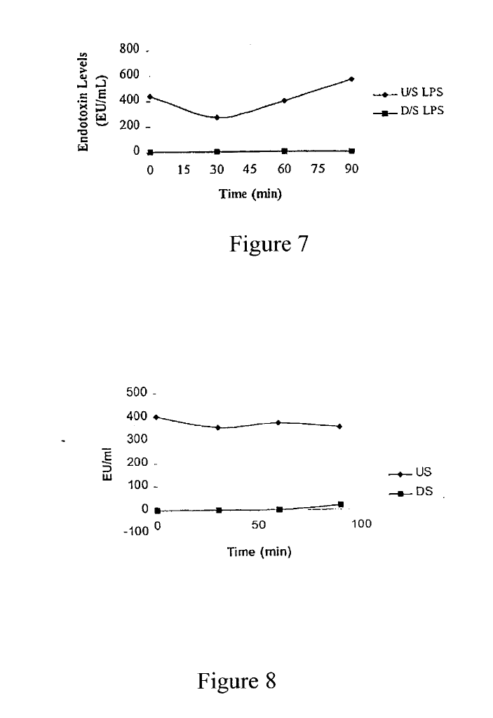

Referring to FIG. 7, samples from the second phase of an IgG separation were taken from both stream 1 and stream 2 (S1 and S2 respectively) at 30 minute intervals. The samples were assayed for endotoxin using a LAL Chromogenic assay (Cape Cod Assoc.)

HSA Purification Procedure

Pooled normal plasma was diluted one in three with Tris-Borate (TB) running buffer, pH 9.0 and spiked with 600 EU/mL of purified endotoxin. The mixture was placed in stream 1 the electrophoresis apparatus. Albumin was isolated from platelet free plasma in a one-phase process using the charge of albumin at a pH above its pI and its molecular weight. Thus, a cartridge having a separation membrane with 75 kDa cutoff and restriction membranes with 50 kDa cutoffs. The albumin was removed from high molecular weight contaminants by its migration through the separation membrane while small molecular weight contaminants dissipated through the 50 kDa restriction membrane. Samples were taken at regular intervals throughout a 180 minutes run.

The presence of the purified HSA in stream 2 was demonstrated by examination by SDS-PAGE. Endotoxin was tested for in both stream 1 and stream 2 samples using a LAL Chromogenic assay supplied by Cape Cod Associates. All samples were appropriately diluted and the endotoxin assay was performed according to the manufacturer instructions.

Referring to FIG. 8, HSA was purified from endotoxin spiked plasma. Samples were taken from up- and stream 2 at 30 minute intervals during a 90 minute purification (x-axis). Analysis of the samples using a LAL Chromogenic assay was performed to establish the endotoxin concentration (y-axis) in the samples.

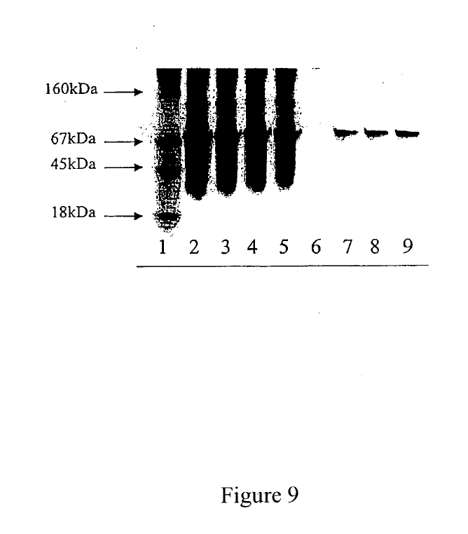

Referring to FIG. 9, 4 to 20% native gel electrophoresis of samples from an HSA purification from endotoxin spiked plasma. Lane 1 contains molecular weight markers, Lane 2 contains starting plasma sample, Lanes 3-5 contain stream 1 samples at time 30, 60, and 90 minutes, Lanes 6-9 contain stream 2 samples at time 0, 30, 60 and 90 minutes, respectively.

Bacteria Removal During Plasma Protein Purification

IgG Purification Procedure

Platelet free plasma was diluted one part in three with Tris-borate, pH 9.0 running buffer and placed in stream 1 of the electrophoresis apparatus and spiked with E. coli to a concentration of 4×10⁸ cells/mL. A potential of 250 V was placed across a cartridge having a separation membrane with 200 kDa cutoff and restriction membranes with 100 kDa cutoffs. A separation membrane of this size restricts IgG migration while allowing smaller molecular weight contaminants to pass through the separation membrane, leaving IgG and other large molecular weight compounds in stream 1. A second purification phase was carried out using a GABA/Acetic acid buffer, pH 4.6 with a cartridge having a separation membrane with 500 kDa cutoff and restriction membranes with 3 kDa cutoffs. A potential of 250 V reversed polarity was placed across the system resulting in IgG migration through the separation membrane leaving other high molecular weight contaminants in stream 1.

Examination of samples taken at 30 minutes intervals was made on reduced SDS-PAGE 4-20% gels. Twenty microliters of stream 1 or 100 μL of stream 2 samples were spread plated onto Luria agar culture plates. The plates were incubated for 24 hours at 37° C., and the number of colonies was counted.

HSA Purification Procedure

Pooled normal plasma was diluted one in three with Tris-Borate (TB) running buffer, pH 9.0 and spiked with approximately 4×10⁸ cells/mL of E. coli. The mixture was placed in stream 1 of the electrophoresis apparatus. Albumin was isolated from platelet free plasma in a one-phase process using the charge of albumin at a pH above its pI and its molecular weight. Thus a cartridge with a 75 kDa cutoff separation membrane and 50 kDa cutoff restriction membranes was used. The albumin was removed from high molecular weight contaminants by its migration through the separation membrane while small molecular weight contaminants dissipated through the 50 kDa restriction membrane.

Samples were taken at regular intervals throughout a 90 minutes run. The presence of the purified HSA in stream 2 was demonstrated by examination by SDS-PAGE. Bacteria were detected as previously described above. The stream 2 samples containing the purified protein products did not contain detectable E. Coli colonies, while stream 1 samples produced greatly in excess of 500 colonies/plate.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for removing a non-enveloped virus from a sample containing a target compound, comprising:
(a) placing the sample in a first solvent chamber of an electrophoresis apparatus having a cathode and an anode, the first solvent chamber being separated from a second solvent chamber by a separation barrier having a defined pore size and disposed between the cathode and anode;
(b) selecting a solvent for the first solvent chamber having a defined pH;
(c) adding a solvent to the second solvent chamber;
(d) applying an electric potential between the cathode and anode whereby at least a portion of the target compound is located on one side of the separation barrier while non-enveloped virus is located on the other side of the separation barrier; and
(e) maintaining step (d) until the desired purity of the target compound is reached on one side of the separation barrier.

2. A method for removing a non-enveloped virus from a sample containing a target compound, comprising:
(a) placing the sample in a first solvent chamber of an electrophoresis apparatus having a cathode and an anode, the first solvent chamber being separated from a second solvent chamber by an separation barrier having a defined pore size and disposed between the cathode and anode;
(b) selecting a solvent for the first solvent chamber having a defined pH;
(c) adding a solvent to the second solvent chamber;
(d) applying an electric potential between the cathode and anode whereby at least a portion of the target compound moves through the barrier into the second solvent chamber while virus is substantially retained in the first solvent chamber or the barrier; and
(e) maintaining step (d) until the second solvent chamber contains a desired purity of the target compound.

3. A method for removing a non-enveloped virus from a sample containing a target compound, comprising:
(a) placing the sample in a first solvent chamber of an electrophoresis apparatus having a cathode and an anode, the first solvent chamber being separated from a second solvent chamber by an separation barrier having a defined pore size and disposed between the cathode and anode;
(b) selecting a solvent for the first solvent chamber having a defined pH;
(c) applying an electric potential between the cathode and anode whereby the virus moves through the barrier into the second solvent chamber while the target compound is substantially retained in the first sample chamber or the barrier;
(d) optionally, periodically stopping and reversing the electric potential; and
(e) maintaining step (c), and optional step (d) until the second solvent chamber has reached the desired purity of target compound.

4. The method according to claims 1, 2 or 3 whereby the non-enveloped virus is selected from the group consisting of immunodeficiency virus, hepatitis virus, adenovirus, reovirus, rhinovirus, papillomavirus, foot and mouth disease virus, or parvovirus, and combinations thereof.

5. The method according to claim 4 whereby the immunodeficiency virus is human immunodeficiency virus 1 or human immunodeficiency virus 2.

6. The method according to claim 4 wherein the hepatitis virus is hepatitis A virus, hepatitis B virus or hepatitis C virus.

7. The method according to claim 4 whereby the parvovirus is Parvovirus B-19.

8. The method according to claims 1, 2 or 3 wherein the target compound is selected from the group consisting of peptide, growth factor, lactalburnin, lactoglobulin, blood protein, immunoglobulin, and recombinant protein.

9. The method according to claims 1, 2 or 3 further including removing a biological contaminant selected from the group consisting of lipopolysaccharide, toxin, endotoxin, bacteria, fungi, yeast, pathogens, infectious agent, and protozoan.

10. The method according to claims 1, 2 or 3 whereby the solvent for the first solvent chamber has a pH lower than the isoelectric point of the target compound.

11. The method according to claims 1, 2 or 3 whereby the solvent for the first solvent chamber has a pH at the isoelectric point of the target compound.

12. The method according to claims 1, 2 or 3 whereby the solvent for the first solvent chamber has a pH above the isoelectric point of the target compound.

13. The method according to claims 1, 2 or 3 whereby the solvent and/or sample are passed through the solvent chambers to form streams.

14. The method according to claims 1, 2 or 3 whereby the membrane has a molecular mass cut-off close to the apparent molecular mass of the target compound.

15. The method according to claims 1, 2 or 3 whereby the membrane has a molecular mass cut-off of at least about 3 kDa.

16. The method according to claims 1, 2 or 3 whereby the membrane has a molecular mass cut-off of between 3 and 1000 kDa.

17. The method according to claims 1, 2 or 3 whereby the electric potential applied is up to about 300 volts.

18. The method according to claims 1, 2 or 3 whereby the target compound is collected or removed from the second solvent chamber.

19. The method according to claim 1 wherein step (e) results in the target compound being substantially free of non-enveloped virus.
